(19) United States
(12) Patent Application Publication
(10) Pub. No.: US 2014/0235483 A1
(43) Pub. Date: Aug. 21, 2014

(54) PARALLEL DETERMINATION OF COMPETITIVE BINDING AND TARGET SPECIFICITY OF A BINDING COMPOUND LIBRARY BY HIGH-THROUGHPUT DNA SEQUENCING

(71) Applicant: Full Spectrum Genetics, Inc., South San Francisco, CA (US)

(72) Inventor: Robert DuBridge, Belmont, CA (US)

(73) Assignee: Full Spectrum Genetics, Inc., South San Francisco, CA (US)

(21) Appl. No.: 14/181,695

(22) Filed: Feb. 16, 2014

Related U.S. Application Data
(60) Provisional application No. 61/766,047, filed on Feb. 18, 2013.

(51) Int. Cl. G01N 33/53

(52) U.S. Cl. CPC G01N 33/5308
USPC 506/9

(57) ABSTRACT

The invention is directed to a method for analyzing the competitive binding and target (or ligand) specificity of large numbers of candidate binding compounds with respect to a predetermined reference compound. That is, the invention provides a method for essentially conducting a massively parallel ELISA on each member of an entire library of candidate binding compounds at the same time. Instead of determining binding characteristics from a series of colorimetric or fluorometric readouts, such characteristics are determined from a series of frequencies of bound and unbound library members which, in turn, are determined by high-throughput sequencing of their encoding nucleic acids. In one aspect, predetermined reference compounds are proteins and candidate binding compounds are members of a mutant library based on or related to, the predetermined reference compound.

This application claims priority from U.S. provisional application Ser. No, 61/766,047 filed 18 Feb. 2013, which is incorporated by reference herein in its entirety.

### BACKGROUND

New generations therapeutic antibodies are being engineered and developed that have a host of performance improvements, including modified affinities, increased stability, reduced immunogenicity, greater solubility, and the like, e.g. Igawa et al, mAbs, 3(3): 243-252 (2011); Bostrom et al, Science, 323: 1610-1614 (2009). An important approach for making such improvements is to create a mutant library from an existing therapeutic antibody then screen library members b various assays until better performing antibodies are found. Because such libraries are typically very large, such screening can be expensive and time consuming unless high throughput tools are available. In particular, the target specificity and non-specific binding characteristics of candidate compounds have been difficult to assess efficiently because of a dearth of high-throughput techniques for this purpose.

Competitive enzyme-linked immunosorbent assays (ELISAs) have been highly useful for determining the binding and specificity characteristics of candidate compounds; however, such assays typically can be run on only a single candidate at a time and attempts to increase the throughput of such assays have resulted in only limited degrees of multiplexing., e.g. Butler, J. Immunoassay, 21(2&3): 165-209 (2000); Kingsmore, Nature Reviews Drug Discovery, 5: 310-320 (2006); Ellington et al, Clin. Chem., 56(2): 186-193 (2010); Yang et al, U.S. patent publication 2009/0123336.

In view of the above, it would be highly advantageous for therapeutic antibody development if techniques were available that permitted convenient high throughput comparisons of binding and specificity characteristics of candidate compounds with those of reference compounds.

### SUMMARY OF THE INVENTION

The present invention is directed to methods for high-throughput measurements of competitive binding and specificity characteristics of libraries of compounds. Aspects of the present invention are exemplified in a number of implementations and applications, some of which are summarized below and throughout the specification.

In one aspect, the invention is directed to a method of simultaneously determining competitive binding and target specificity of binding compounds of a library relative to a predetermined reference compound. In one embodiment such method comprises the following steps: (a) reacting under binding conditions in a plurality of reactions a predetermined reference compound and a library of binding compounds with a ligand, wherein the predetermined reference compound is present in each reaction at a different concentration and wherein each binding compound is a protein encoded by a linked nucleic acid; (b) separating in each reaction binding compounds forming complexes with the ligand from binding compounds free of ligand; (c) sequencing for each reaction nucleic acids of a sample of binding compounds forming complexes with the ligand to determine frequencies of the nucleic acids; (d) sequencing for each reaction nucleic acids of a sample of expressed binding compounds free of ligand or a sample of expressed binding compounds from the library to determine frequencies of the nucleic acids; (e) determining affinities of the binding, compounds of the samples from each reaction, wherein the affinities are determined by comparing the frequency of nucleotide sequences identified with a binding compound forming complexes with the ligand and the frequency of the same nucleotide sequences identified with the binding compound free of the ligand or with the frequency of the same nucleotide sequences that encode the same binding compound in the library; and (f) identifying a binding compound as competitively binding with the predetermined reference compound to the ligand whenever the affinities of the binding compound are a monotonically decreasing function of predetermined reference compound concentration, or identifying a binding compound as non-competitive with the predetermined reference compound in binding to the ligand whenever the affinities of the binding compound are a substantially unchanging function of predetermined reference compound concentration. In some embodiments, the step of separating may be facilitated by attaching either the ligand or the predetermined reference compound to a solid support. In the latter embodiment, the ligand is presented in different concentrations in the different reactions.

These above-characterized aspects, as well as other aspects, of the present invention are exemplified in a number of illustrated implementations and applications, some of which are shown in the figures and characterized in the claims section that follows. However, the above summary is not intended to describe each illustrated embodiment or every implementation of the present invention.

### BRIEF DESCRIPTIONS OF THE DRAWINGS

FIG. 1A diagrammatically illustrates the general concept of one embodiment the invention.

FIG. 1B is a diagram of a work flow for an embodiment of the invention in which nucleic acids encoding a library of binding compounds is sequenced and nucleic acids encoding members of the library that bind to targets is sequenced.

FIG. 1C is a diagram of a work flow for another embodiment of the invention in which nucleic acids encoding binder and nonbinders are sequenced.

FIG. 2 illustrates an alternative bead-based method for isolating from a solution phase reaction ligands having reference or candidate compounds bound.

FIG. 3 is a diagram of an immunoglobulin G molecule and its constituent regions.

FIGS. 4A and 4B show exemplary data from a competitive binding reaction between Avastin and mutant binding compounds having amino acid substitutions at the F100f location of the heavy chain.

### DETAILED DESCRIPTION OF THE INVENTION

The practice of the present: invention may employ, unless otherwise indicated, conventional techniques and descriptions of organic chemistry, molecular biology (including recombinant techniques), cell biology, and biochemistry, which are within the skill of the art. Such conventional techniques include, but are not limited to, preparation of synthetic polynucleotides, monoclonal antibodies, antibody display systems, nucleic acid sequencing and analysis, and the like. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV); PCR Primer; A Laboratory Manual; Phage Display: A Laboratory Manual; and Molecular Cloning: A Laboratory Manual (all from Cold Spring Harbor Laboratory Press); Sidhu, editor, Phage Display in Biotechnology and Drug Discovery (CRC Press. 2005); Lutz and Bornscheuer, Editors, Protein Engineering Handbook (Wiley-VCH, 2009); Hermanson, Bioconjugate Techniques, Second Edition (Academic Press, 200); Wild, The Immunoassay Handbook, Third Edition (Elsevier Science), and the like.

The invention is directed to a method for analyzing the competitive binding and target (or ligand) specificity of large numbers of candidate binding compounds with respect to a predetermined reference compound. That is, the invention provides a method for essentially conducting a massively parallel ELISA on each member of an entire library of candidate binding compounds at the same time. However, instead of determining binding characteristics from a series of colorimetric or fluorometric readouts, such characteristics are determined from a series of frequencies of bound and unbound library members which, in turn, are determined by high-throughput sequencing of their encoding nucleic acids. In some embodiments, encoding nucleic acids are linked to their proteins so that steps depending on a binding compound's affinity and/or specificity, such as separations, partitions, isolations, or the like, operate on both the binding compound and its encoding nucleic acid. In some embodiments, such linking of binding compounds and their encoding nucleic acids is carried out by the use of protein display systems as binding compounds.

In one aspect, predetermined reference compounds are proteins and candidate binding compounds are members of a mutant library based on, or related to, the predetermined reference compound. Typically a library derived from the predetermined reference compound is made by introducing a variety of changes to the predetermined reference compound, including, but not limited to, one or more substitutions of amino acids at one or more locations, or a plurality of locations. In alternative embodiments, changes may include deletions and/or insertions of amino acids. Generating such libraries is well-known in the art and is disclosed in the following references that arc incorporated by reference: DaBridge, U.S. patent publications 2012/0077691 and 2012/0258866, and the like. In one aspect, the predetermined reference compound is an antibody or a fragment thereof and the library is a protein display system, such as a phage display system, wherein each member binding compound is encoded by a nucleic acid carried by the display organism of the protein display system. Target molecules or ligands may be any compound a protein is capable of binding to. More specifically, a ligand or target molecule may be an organic or inorganic compound, including, but not limited to, a drug or a biomolecule, such as a protein, carbohydrate, peptide, lipid, hormone, receptor, or the like.

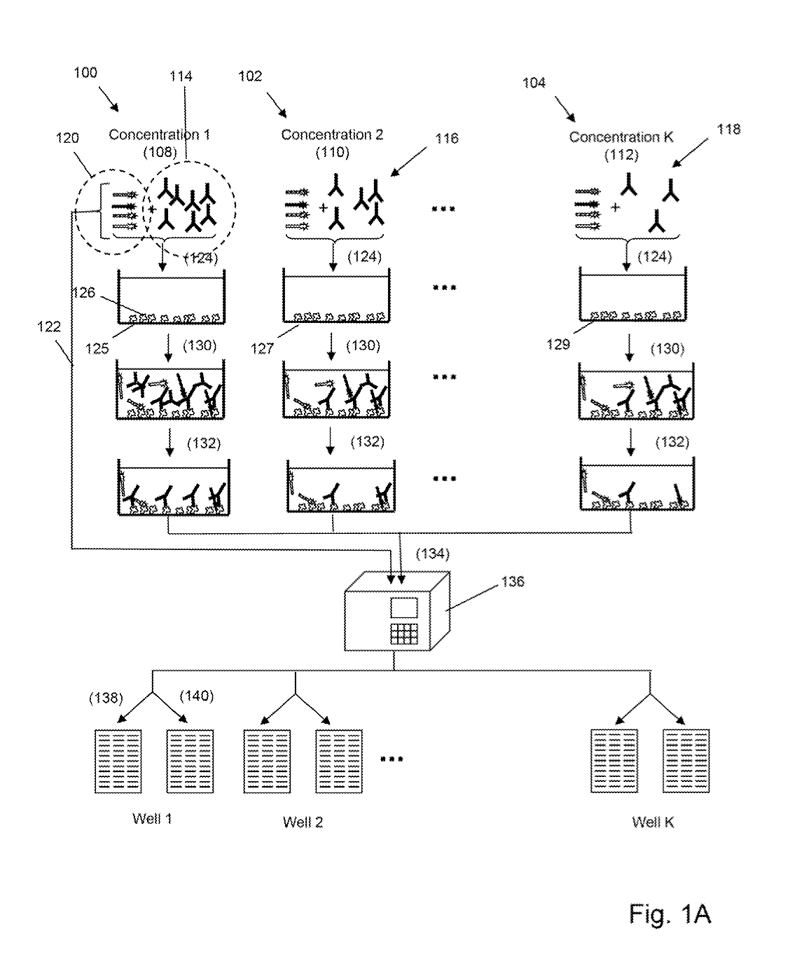

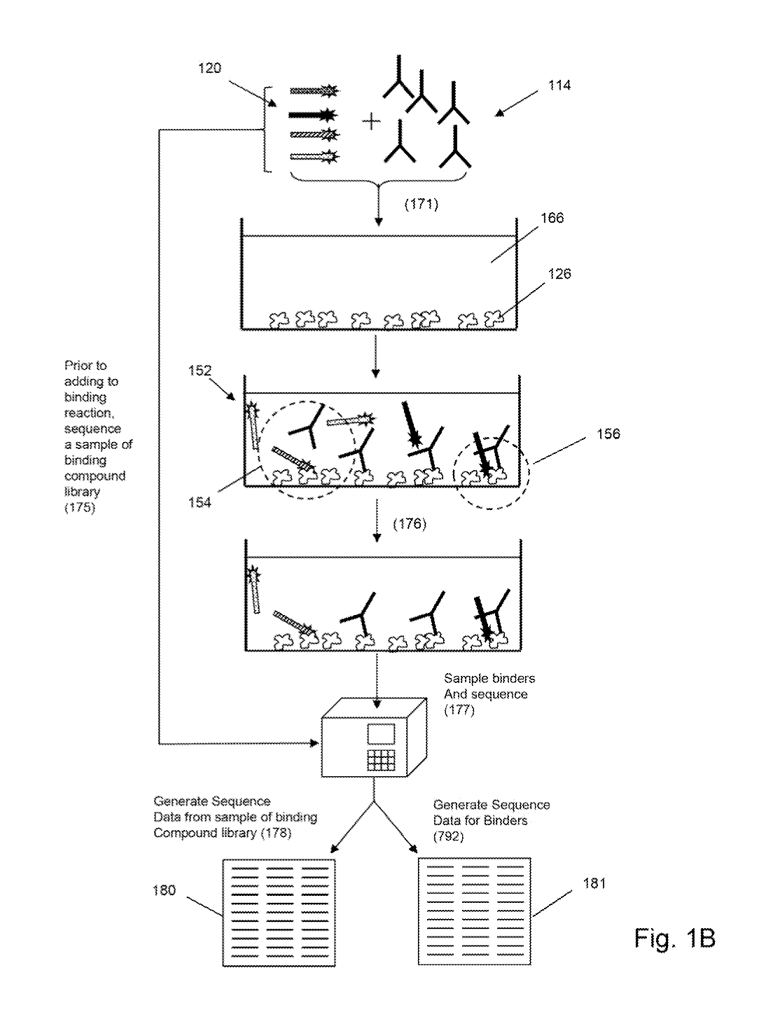

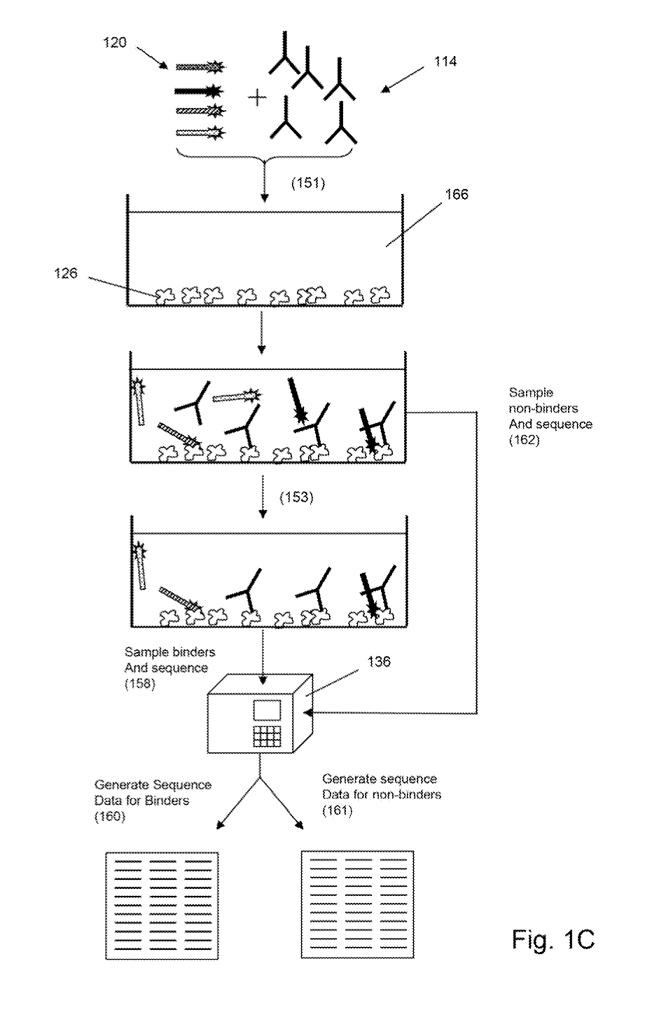

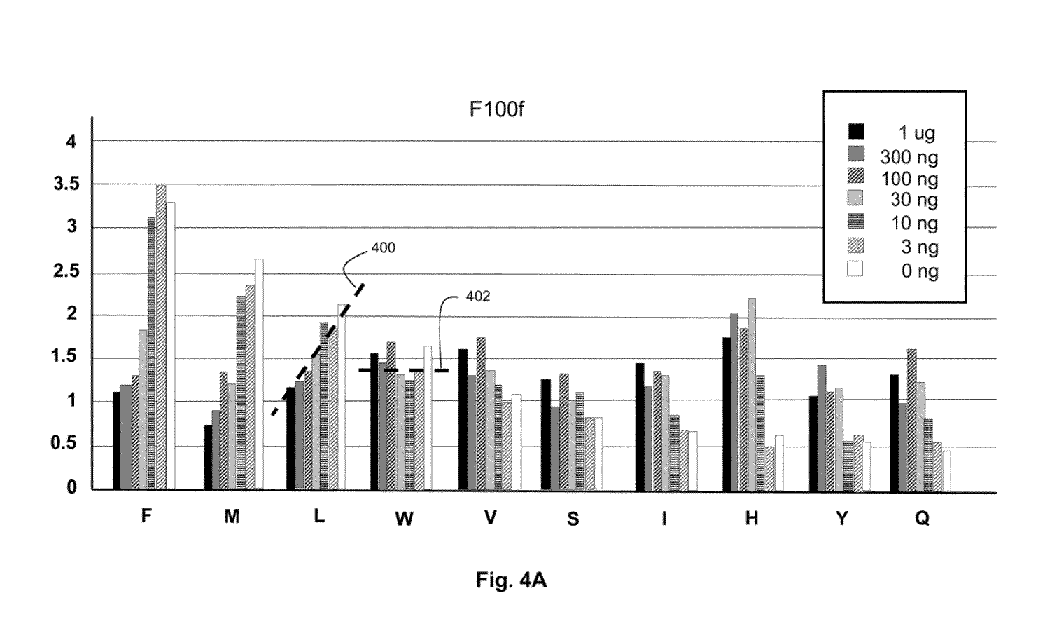

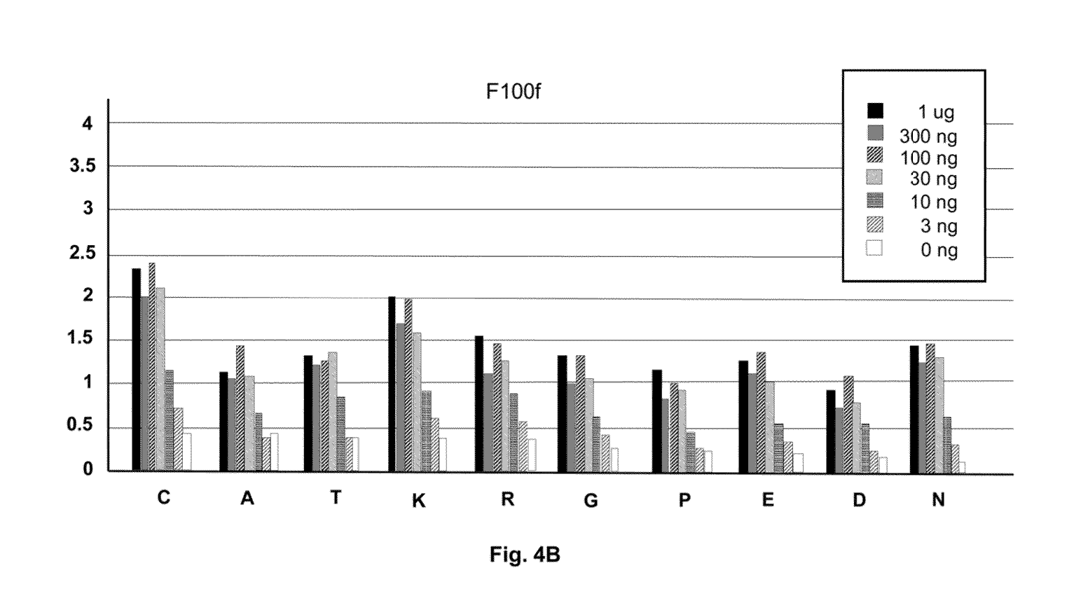

FIG. 1A provides an overview of one embodiment of the invention. Prior to carrying out binding reactions, a sample of binding compound library (120) is taken and sequenced (122) so that frequencies of operational binding compounds are determined. (As explained more fully below, in some protein display systems inactive compounds are expressed which may lead to incorrect affinities unless removed or otherwise taken into account). Bottoms of a series of wells 1, 2, . . . K, (125, 127, and 129, respectively) are coated with target molecule, or ligand, (126), after which binding compound library (120) and different concentrations (108, 110, and 112, respectively) of predetermined reference compound (114) are added (124) under binding conditions, e.g. of the predetermined reference compound. After incubation (130) to permit the binding reactions to reach equilibrium, the reaction mixture is removed and the wells are washed (132). It is noted that in accordance with the invention, many configurations of competitive reactions are possible. For example, a reference compound may be attached to a surface (such as a well bottom) and members of a binding compound library may compete with a ligand or target molecule in free solution for binding to the immobilized reference compound. Remaining binding compounds are eluted, their relevant encoding nucleic acids isolated and sequenced (134) using a commercially available high throughput DNA sequencer (136). Sequence data from the different wells may be tracked (e,g. by tag sequences) or kept separate for analysis so that the relative affinities for each binding compound can be associated with the different concentrations of the predetermined reference compound. Thus, for each well pairs of sequence data (138 and 140) are produced. In this embodiment, one member of each pair is a tabulation of binding compound frequencies in the library prior to the reactions. From such sequence data, the effect of the different concentrations of the predetermined reference compound on the amount of each library member retained in a well after washing (132) may be computed. Exemplary data from such computations is illustrated in FIG. 4 (described more fully below). The plurality of reactions (e.g. the number, K, of wells described above) may vary widely. In some embodiments, such plurality is in the range of from 2 to 20; in other embodiments, such plurality is in the range of from 4 to 10. In some embodiments, such plurality is selected to take advantage of standard labware, such as conventional 96-well microwell plates; thus, in some embodiments, a plurality of reactions may be in the range of from 6 to 12.

As mentioned above, competitive assays of the invention may have alternative formats such that any two of the three types of compounds in the reaction may compete with one another to bind to a third. One type is a nucleic acid-encoded mutant library, one type is a predetermined reference compound and one type is a ligand of the reference compound. Two of the three kinds of compound are present in a plurality of reactions in constant amounts or concentrations, while the third is present in different concentrations. In all cases, after binding and formation of stable complexes, mutant library members in such complexes are isolated and sequenced and relative affinities are determined from frequencies of encoding sequences. As illustrated in FIGS. 1A-1C in some embodiments, a ligand is attached to a solid support is equal amounts or concentrations in the different reactions, a mutant library is added to each in equal amounts or concentrations (or at least in known amounts or concentrations), and a predetermined reference compound is added in different amounts or concentrations. In some embodiments, a predetermined reference binding compound is attached to a solid support in equal amounts in the different reactions, and the mutant library is added in equal amounts or concentrations, and the ligand is added in varying concentrations or amounts. In some embodiments, a predetermined reference binding compound is attached to a solid support in equal amounts in the different reactions, and the ligand is added in equal amounts or concentrations, and the mutant library is added in varying concentrations or amounts In some embodiments, a mutant library is attached to a solid support in equal amounts in the different reactions, and the predetermined reference binding compound is added in equal amounts or concentrations, and the ligand is added in varying concentrations or amounts. In some embodiments, a mutant library is attached to a solid support in equal amounts in the different reactions, and the ligand is added in equal amounts or concentrations, and the predetermined reference binding compound is added in varying concentrations or amounts,

shows a more detailed view of the reactions of FIG. 1A. A sample of library (120) is taken and nucleic acid encoding expressed binding compounds is sequenced (175) so that the frequencies of each operable binding compound in the library may be determined. Library (120) is combined (171) with predetermined reference compound (114) in reaction mixture (116) in well (170) that has ligand (126) adsorbed on its bottom surface. Members of library (120) may have a wide range of binding characteristics. For example, some mutants may have enhanced non-specific binding (152), others may compete directly with and displace the predetermined reference compound (156), and others may be specific for a different part of the target (156). After incubation, unbound material is removed, well (170) is washed (176) and remaining material is eluted, after which a segment of the binding compound-encoding DNA is amplified. The amplified segment is selected to have sufficient diversity so that each segment sequence can be unequivocally associated with a single binding compound. For antibody binding compounds, typically segments are selected that encompass at least a portion of a recombined region of an immune receptor chain, such as a CDR segment.

FIG. 1C shows a more detailed view of an alternative embodiment in which binders and non-binders of each reaction are sequenced. As above, library (120) and predetermined reference compound (114) are added to reaction mixture (166) in a well whose bottom surface coated with target molecule (126). After incubation as above, a sample is taken (162) of reaction mixture (166) (which contains expressing unbound library members), after which DNA is extracted and an identifying segment is amplified and sequenced. Also after such sampling, the remaining unbound material is removed and the well is washed (153), after which a sample (158) of bound material is eluted, and DNA extracted and amplified for sequencina. As above, a pair of sequence data is produced (160 and 161) listing encoding nucleic acid sequences of binders and non-binders, respectfully, from which relative affinities may be determined.

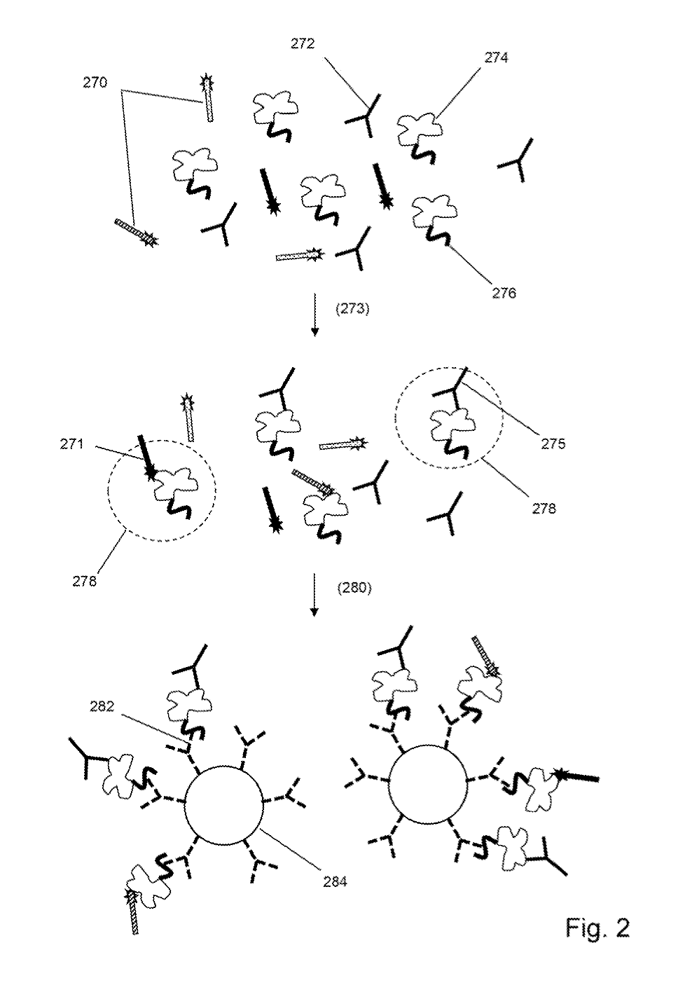

Although the both of the embodiments above show ligand adsorbed to a surface of a reaction container, the invention is not restricted to that feature. Any available method for separating ligand with bound library members from unbound library members may be used in the practice of the invention. For example, a humid or target molecule may be derivatized with a capture moiety, such as a biotin, an amino acid tag, or the like, which may be used with a complementary agent that is capable of specifically reacting with a capture moiety. The complementary agent may be attached to a bead which then permits unbound material to be separated from the bound material. Such an embodiment is illustrated in FIG. 2. There members (270) of a binding compound library and reference binding compound (272) compete in free solution to bind ligand (274) having capture moiety (276). After incubation under binding conditions (273), some members (271) and some reference binding compound (275) forms stable complexes (278) which may be separated (280) from unbound or uncomplexed compounds by reacting capture moiety (276) with a complementary agent (282) that may be attached to a solid support, such as bead (284). After washing, members (271) from complexes (278) attached to beads (284) may be eluted and analyzed as described above.

In one aspect the embodiments of FIGS. 1B and 1C for identifying binding compounds that have equivalent or improved affinities to a ligand as that of reference binding compound may be carried out with the following steps: (a) reacting under binding conditions a ligand with a library of binding corn minds in solution phase, the library having a size and comprising candidate binding compounds and a reference binding compound, wherein each candidate binding compound and the reference binding compound comprise a protein display system expressing a protein encoded by a nucleotide sequence; (b) sequencing the nucleotide sequences of a representative sample of binding compounds forming complexes with the ligand; and (c) sequencing the nucleotide sequences of a representative sample of binding compounds free of ligand or a representative sample of binding compounds from the library. Further steps that may be implemented with the sequence data from steps (a) to (c) include the following for determining relative affinities of the binding compounds: (d) ordering the nucleotide sequences of the binding compounds in accordance with the affinities of their respective binding compounds for the ligand, wherein the affinities are determined for each binding compound by comparing a number of times a nucleotide sequence is identified with the binding compound forming complexes with the ligand and a number of times the same nucleotide sequence is identified with the binding compound free of the ligand or with the binding compounds of the library; and (e) identifying among the ordering of nucleotide sequences those nucleotide sequences that encode candidate binding compounds having affinities that are equivalent to or greater than that of the nucleotide sequence encoding the reference binding compound. In some embodiments, candidate binding, compounds of the library have from 1 to 2 amino acid substitutions with respect to the reference binding compound wherein each of the substitutions is selected from 0 to 250 amino acid locations in the reference binding compound or 1 amino acid substitution with respect to the reference binding compound wherein each of the substitutions is selected from 1 to 500 amino acid locations in the reference binding compound. In other embodiments, a representative sample contains a number of binding compounds at least five times the size of the library: and in another embodiment, a representative sample contains a number of binding compounds at least ten times the size of the library.

As discussed more fully below, sizes of the candidate compound libraries arc selected so that values of properties of interest, such as the relative affinities, of the library members may be determined with coefficients of variation (CVs) of twenty percent (20%) or less, or in some embodiments with CVs of ten percent (10%) or less, or in some embodiments, with CVs of five percent (5%) or less. Typically such libraries have sizes in the range of from a thousand (1000) members to a few tens of thousands members (e.g. 10,000-50,000 members). In other embodiments, such libraries have sizes in the range of from 1000 to 100,000 members: and in still other embodiments, such libraries have sizes in the range of from 1000 to 1,000,000 members.

### Relative Affinities

In order to obtain reliable statistics on the frequencies of binders and non-binders or frequencies of binding compounds in a library, samples must be sufficiently large to avoid aberrant results due to sampling error. The appropriate sample size depends at least (i) on the degree of reliability desired in determining the proportions of each binding, compound bound or unbound, and (ii) the size of the library of different nucleic acid-encoded binding compounds. Unlike conventional libraries of binding compounds, where maximal diversity is sought, in some embodiments of the present invention, libraries of limited size are employed so that reliable statistics on the binding characteristic of each binding compound can be readily obtained. The size of a library for use with the invention depends on how many residues are varied in the library members, or candidate binding compounds; in other words, the size depends on the number of amino acid positions where amino acids are varied and the number of different amino acids that are substituted in at each such position.

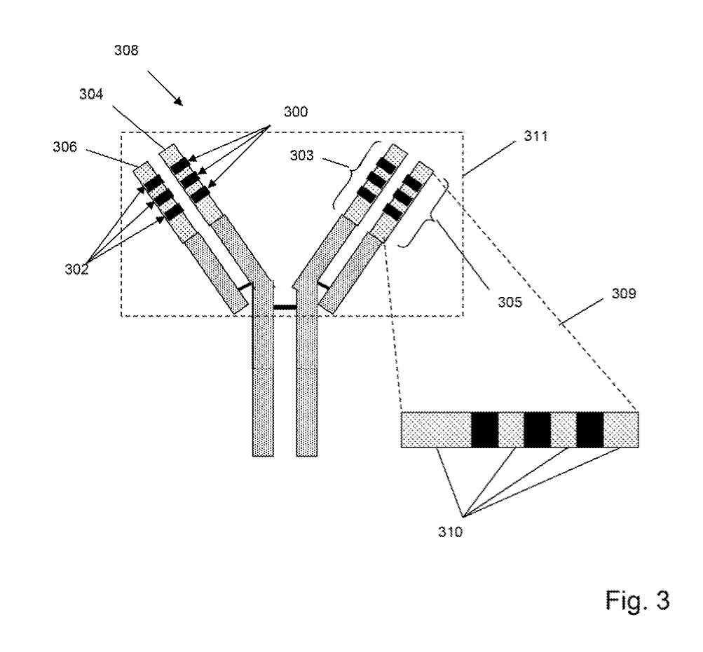

For antibodies, varying the amino acids occupying each amino acid position one at a time in a collection of six complementary determining regions (CDRs) leads to about 1600-2200 library members (where “library” here is in reference to the encoded binding compounds, as opposed to the nucleic acids that are translated into ammo acids, which of course will be more numerous because of the degeneracy of the genetic code), (FIG. 3 illustrates CDRs (black regions, 300) of heavy chain variable region (304 and indicated as 303 in the right hand heavy chain) and CDRs (302) of light chain variable region (306 and indicated as 305 in the right hand light chain) of antibody (308), which has Fab fragment encompassed. by dashed rectangle (311). “Scaffold” or “framework” portions (310) surrounding the CDRs are shown on projection (309) of light chain variable region (305)). In some embodiments of the invention, samples of binders and non-binders for sequencing include many times this number of candidate binding compounds. In sonic embodiments, sample sizes are in the range of about 5 times or more times the library size. In some embodiments, sample sizes are in the range of from about 5 to 100 times the library size. For a 2000 member library of candidate binding compounds, a sample size of in the range of 10⁴-2×10⁶ may be used, for example. For a library containing about 2.3×10⁴ members (e,g,, amino acids of 6 CDRs varied two at as time), a sample size in the range of from 1.1×10⁵ to 2.3×10⁴ may be used. In some embodiments, nucleic acid sequences from such samples are further amplified in the course of sequence analysis. For example, if an illumina GA DNA sequencer is employed, primer binding sites are attached to sequences from such samples in a PCR which allows bridge PCR for forming clusters on a solid phase surface, which are analyzed by the Solexa-based sequencing chemistry. Preferably, multiple copies (e.g. ≧10 copies) of each sequence from such samples are analyzed to ensure reliable sequence determination. Thus, if a sample size of 10⁴ to 2×10⁵ is used then for Solexa-based sequencing, or equivalent technology, at least 10⁵ to 2×10⁶ clusters are formed, or sequence reads obtained, for data analysis; or if a sample size of 10⁵-10⁶ is used then for Solexa-based sequencing, or equivalent technology, at least 10⁶-10⁷ clusters are formed, or sequence reads obtained, for data analysis. In some embodiments, sufficiently large samples are taken so that the measured frequencies have P-values of 0.1 or less, or P-values of 0.05 or less, or P-values of 0.002 or less. In alternative embodiments, nucleic acids encoding scaffold regions may also be used to generate library members either by selective amino acid substitutions, additions, and/or deletions, or by substitution of scaffolds or frameworks from different antibodies, e.g. from different individuals.

Hosts expressing binding compounds are readily separated from non-expressing hosts using antibodies specific for constant regions, e.g. goat anti-kappa chain antibody for isolating phage expressing human Fab fragments.

From the sequence data of the embodiment of FIG. 1B, frequencies of each binding compound in the library can be computed and frequencies of each binding compound that actually binds to a ligand in a reaction can be computed. Those binding compounds with the highest percentage of bound display host (e.g. phage) will have the highest affinities and those with the lowest bound percentages will have the lowest affinities. Using a single ligand concentration near the dissociation constant, K_{D}, of the parent protein. It is possible to rank the affinities every protein variant for a given ligand. If the parent molecule is encoded in the library, then the affinities of all of the variants in the library can be assessed relative to the parent protein, which serves as an internal standard or reference. If the ligand is in great excess in the binding reaction (so its unbound concentration does not change appreciably during the binding reaction) and several binding reactions are run using varying ligand concentrations, then one is able to use non-linear regressions or equivalent calculation to rapidly calculate the K_{D} for every variant in the population from the equation K_{D}=[A][B];[AB], where [A] is the concentration of a first member of a binding pair in the unbound state, [B] is the concentration of a second member of a binding pair in the unbound state, and [AB] is the concentration of the first and second members in the bound state. In some embodiments employing protein display systems, such as phage display libraries, some properties of interest, such as affinities, may be estimated as follows based on tabulated sequences of nucleic acids encoding binding compounds. For example, for measuring relative affinities, multiple reactions are set up, e.g. In wells of a microtiter plate, or the like, such that the reactions contain a dilution series of ligand, i.e., a series of lower and lower concentrations or amounts of ligand adsorbed or attached to a solid support, such as the surface of microwell wall, magnetic bead, or the like. To each reaction is added a fixed number of display organism, such as aliquots of a phage display library, and the reactions are allowed to go to equilibrium. After equilibrium has been reached, bound and free display organisms are harvested and binding-compound encoding nucleic acids are amplified in separate polymerase chain reactions (PCRs) to determine the reaction in which the concentration, or amount, of ligand results in about equal amounts of display organism bound ligand and free. Under such conditions, affinities of the binding compounds may be estimated as ratios abound binding compound (determined by counting encoding nucleic acids) and unbound binding, compound (also determined by counting encoding nucleic acids).

In some embodiments, a similar operation may be used to estimate affinities of binding compounds of a library relative to that of a reference binding compound (as used herein, such values are referred to as “relative affinities” with respect to a selected reference compound). As above, multiple reactions are set up with a dilution series of immobilized ligand. To each reaction is added a fixed amount of reference binding compound (e.g. a single phage displaying the reference binding compound) and the reactions are allowed to go to equilibrium. After equilibrium has been reached, bound and free display organisms are harvested and their encoding nucleic acids are amplified in separate PCRs to determine the reaction in which the concentration, or amount, of ligand results in about equal amounts of reference binding compound bound to ligand and free of ligand. The determined reaction provides conditions for carrying out library-based binding reactions so that ratios of binders to nonbinders for each library member can be computed and compared to that of a reference binding compound to give a measure of the relative affinity of such member to a ligand.

In one aspect, statistically significant information is obtained about how structural elements of proteins; e,g. position and identity of amino acid residues in binding domains, relate to functional properties of interest, such as binding affinity, specificity, expression, stability, cross-reactivity, and the like. As mentioned above, such information is collected by identifying sufficient numbers of binding compounds that are segregated or separated into subsets in selection reactions. For example, for relative affinities, such information may be collected by reacting under binding conditions a set of candidate nucleic acid-encoded binding compounds with one or more target molecules, so that complexes form between the one or more target molecules and at least a portion of the candidate binding compounds (referred to herein as “binders”). Sufficient numbers of candidate binders and non-binders are then decoded by high throughput nucleic acid sequencing to give statistically significant data about the binding properties of substantially all the members of the set of candidate binding compounds. In other words, sample sizes are large enough so that the numbers of candidate binders and non-binders decoded and recorded are subject to minimal sampling error. As mentioned above, in some embodiments, statistically significant values of properties of interest (such as, relative affinity, expression level, stability, and the like) are obtained when such values are measured with coefficients of variation (CVs) of twenty percent (20%) or less, or in some embodiments with CVS of less than or equal to ten percent; or in other embodiments, with CVs less than or equal to five percent; or in other embodiments, with CVs less than or equal to two percent. Whenever degenerate codons are used when synthesizing candidate binding compounds, CVs of properties of interest, such as relative affinity, or the like, are readily determined from the numbers of sequences counted which contain different codons that encode the same amino acid. For example, if candidate binding compounds of a library are generated from nucleotide sequences each containing a degenerate codon, “NNN”, at each of a number of different sites, then at each such site there will be, for example, six codons encoding serine, four codons encoding valine, and so on. If the expression of the candidate binding compounds is assumed to be uniform, then the degree of deviation from equal representation of each alternative serine codon, or valine codon, or the like, among the enumerated sequences in a selection assay is related to, and a direct measure of, the CV of the encoded binding compound. That is, such deviation is a measure of sampling error, which may be reduced by increasing the number of nucleotide sequences analyzed for a given library and selection assay. In one aspect of the invention, CVs of values of properties of interest (e.g. relative affinities, expression levels, stabilities, cross-reactivities and the like) are conveniently estimated from the CVs of counts of nucleotide sequences with synonymous codons that encode the same binding compound. In one embodiment, such CVS are estimated based on amino acids having from four to six synonymous codons.

The statistically significant information is contained in the tabulations of the sequences of nucleic acids encoding the binders and the non-binders in the different selection reactions. Nucleic acid-encoded binding compounds may be obtained from the various antibody display techniques, aptamers, or the like, such as those described below. In some embodiments, the structural elements that are analyzed are spatially local in the sense that they exert their effects on binding within or near a limited volume of a larger molecule, such as, an enzyme active site, antibody binding site, complementary-determining regions, or the like. In particular, structural elements analyzed in art antibody binding interaction includes CDRs as well as framework regions of antibody variable regions. Alternatively, such information may be collected by first decoding the sequences of members of the total effective library of candidate nucleic acid-encoded binding compounds, for an adequate sample thereof to ensure nearly complete coverage (e.g. at least 95, or at least 98%, or at least 99% coverage)), prior to carrying out a binding reaction with the one or more target molecules, or ligands. As used herein, “total effective library” means the total library of nucleic acid-encoded binding compounds, subject to any biases in sequence representation that may arise in the course of expression, e.g. In phage, ribosomes, bacteria, yeast, or the like. A binding reaction is carried out as described above, after which the nucleic acid sequences of only the binders are determined. From this information, a ratio may be formed for each candidate nucleic acid-encoded binding compound that consists of the number of sequence reads among the binders over the number of sequence reads in the total library as a measure of its binding strength or affinity. That is, the larger the value of the ratio of a candidate binding compound, the stronger its affinity for the one or more target molecules and the lower the value of the ratio the lower its affinity. Generally, such ratios and other ratios, such as ratios of binders to nonbinders, provide relative affinities of each of the binding compounds in the reaction with the one or more ligands.

### Protein Display Systems

Features of any peptide or protein display system are; 1, Tight linkage between the expressed proteins and their encoding nucleic acid: and 2, Expression of the protein in a format that allows it to be assayed and separated based on some biochemical activity (for example, binding strength, susceptibility to enzymatic action, or the like). As used herein, “tight linkage” or simply “linked” in reference to expressed proteins and their encoding nucleic acid means the two molecules are part of the same physical or biological entity, such as, a protein expressed on the surface of a cell that contains the nucleic acid encoding such protein. Alternative physical and biological entities having such tight linkage includes beads or nanoparticles, phages, yeast cells, bacterial cells, mammalian cells, ribosomes, and the like. For the purposes of this discussion, protein display systems can be separated into two groups based on the number of displayed proteins per display unit, either polyvalent or monovalent. The polyvalent display systems such as yeast display (references 1 and 2 below), mammalian display systems (references 3 and 4 below) and bacterial display systems (reference 5) express the gene(s) of interest (often diverse antibody libraries) as proteins tethered to the cell surface by means of a membrane anchor, similar to a native surface immunoglobulin found on the plasma membrane of normal B-cells. DNA encoding the library clones is transformed into the cell type of interest such that each cell receives at most one clone from the library. The resultant population of cells will each express tens to tens of thousands of copies of a single protein clone on their cell surfaces. This population of cells can then be exposed to limiting amounts of fluorescently labeled target antigen and the best binding clones will bind the most antigen and they can be identified and isolated using a fluorescence-activated cell sorter (FACS). Unfortunately accurate quantitation in polyvalent display systems is complicated by cooperative binding effects (avidity) between the multiple copies of the displayed molecule on the same cell (reference 6). This problem is especially pronounced if the antigen is polyvalent (TNF, IgG) or bound to a cell surace (e.g. CD 20).

Many of the viral and phage-based protein display systems arc also polyvalent in nature, but the display units are too small to detect on the FACS, so accurate quantitation is even more difficult. These systems also suffer from avidity problems if multiple binding compounds are expressed simultaneously on the same phage particle. Under such conditions it is difficult to determine whether an observed binding strength is due to the combined effect of two expressed binding compounds versus the effect of a single very high affinity binding compound. Such avidity problems may be minimized by regulating the expression of candidate binding compound in a host using conventional techniques. In one embodiment in which a phage display system expresses Fab fragments, e.g., as disclosed in FIG. 5, regulation of Fab expression is adjusted so that the fraction of phage expression Fab is in the range of from about 0.002 to 0.001, or in the range of about 0.001 to 0.0005.

The monovalent phage (reference 7) and viral (reference 8) systems, along with the ribosome display systems (references 9 and 10) express an average of ≦1 molecule of the displayed molecule per display unit. These systems yield accurate measurements of the true affinity of the binding site in question for each clone in the library. Generally these systems are used to display large, diverse libraries of binding elements. Small subpopulations of clones are then selected from these libraries based on their increased ability to bind the target antigen relative to other members of the library. After selection (often multiple rounds of selection) the resultant clones are isolated and characterized (e.g. as disclosed in U.S. Pat. No. 7,662,557 which is incorporated herein by reference). This is a good strategy for isolating initial binders to a given target antigen from a very large and diverse library, but is not an efficient method for mapping a single protein binding site for the purposes of protein engineering. To achieve this goal one would like to characterize the effect of every possible engineering change and then design and construct an optimized binding site based on: affinity, stability, cross-reactivity, immunogenicity, circulating half-life, manufacturing yield, etc. Therefore it would be desirable to analyze the binding strength of every member of a saturated single substitution library of the binding site in question. The above protein display techniques are disclosed in the following exemplary references, which are incorporated herein by reference; (1) Wittrup, K D Current Opinion in Biotechnology 12: 395-399 (2001) (Protein engineering by cell-surface display); (7) Lauren R. Pepper, Yong Ku Cho, Erie T. Boder and Eric V. Shusta; Combinatorial Chemistry & High Throughput Screening 11: 127-1:44 (2008); (3) Yoshiko Akamatsu, Kanokwan Pakabunto, Zherighai Xu, Yin Zhang, Naoya Tsurushita; Journal of Immunological Methods 327: 40-52 (2007); (4) Chen Zhou, Frederick W. Jacobsen, Ling Cai, Qing Cheri and Weyen David Shen; mAbs 2(5): 1-11 (2010); (5) Patrick S Daugherty; Current Opinion in Structural Biology 17:474-480 (2007) (Protein engineering with bacterial display); (6) Clackson and Lowman (editors), Phage Display (2009); (7) Hennie R Hoogenboom Andrew D Griffiths; Kevin S Johnson, David J Chiswell, Peter Hudson and Greg Winter; Nucleic Acids Research 9(15): 4133-4137 (1991); (8) Francesca Gennari, Luciene Lopes, Els Verhoeyen, Wayne Marasco, Mary K. Collins; Human Gene Therapy 20: 554-562 (2009); (9) Christiane Schaffitzel, Jozef Hanes, Lutz Jermutus, Andreas Pluckthun; Journal of Immunological Methods 231: 11 9-135 (1999) (ribosome display); (10) Robert A Irving, Gregory Coin, Anthony Roberts, Stewart D Nuttall, Peter J Hudson; journal of Immunological Methods 248: 31-45 (2001) (ribosome display); (11) Arvind Rajpal, Nurten Beyaz, Laurie Haber, Guido Cappuccilli, Helena Yee, Ramesh R Bhatt, Toshihiko Takeuchi, Richard A Lerner. Roberto Crea; PNAS 102 (24): 8466-71(2005). Some of the above techniques are also disclosed in the following patents, which are incorporated herein by reference: U.S. Pat. Nos. 7,662,557; 7,635,666; 7,195,866; 7,063,943; 6,916,605; and the like,

Further protein display systems for use with the invention include baculoviral display systems, adenoviral display systems, lentivims display systems, retroviral display systems, SplitCore display systems, as disclosed in the following references: Sakihama et al, PLosOne 3(12): e4024 (2008); Makela et al, Combinatorial Chemistry & High Throughput Screening, 11: 86-98 (2008); Urano et al, Biochem. Biophys. Res Comm., 308: 191-196 (2003); Gennari et al, Human Gene Therapy, 20: 554-562 (2009); Taube et al, PLosOne, 3(9): c3181 (2008): Urn et al, Combinatorial Chemistry & High Throughput Screening, 11: 111-117 (2008); Urban et al, Chemical Biology. 6(1): 61-74 (2011); Buchholz et al, Combinatorial Chemistry & High Throughput Screening, 1: 99-110 (2008); Walker et al, Scientific Reports, 1(5): (14 Jun. 2011); and the like.

In sonic embodiments, the invention employs conventional phage display systems for Improving one or more properties of an antibody binding compound., particularly a preexisting antibody binding compound. Unlike prior applications of display technologies, which employ repeated cycles of selection, washing, elution and amplification, to identify individual phage from a large library, e.g. >10”-1(P clones, in the present invention, a single equilibrium binding reaction is created using a relatively small and focused library, e,g. 10³-10⁴ clones, or in some embodiments 10⁴-10⁵ clones, after which hinder and non-binders are analyzed by large-scale sequencing. From such analysis, subsets are selected and, optionally, further selected based on other properties of interest, such as, solubility, stability, lack of immunogenicity, and the like. Factors affecting such equilibrium reactions are well-known in the art and include: the number of phage to include in the reaction, the stringency of the reaction mixture; the number of target molecules to include in the reaction; presence or absence of blocking agents, such as, bovine serum albumin, gelatin, casein, or the like, to reduce nonspecific binding; the length and stringency of a wash step to separate non-binders; the nature of an elution step to remove binders from the target molecules; the format of target molecules used in the reaction, which, for example, may be bound to a solid support or derivatized with a capture agent, e.g. biotin, and free in solution: the phage protein into which candidate binding compounds are inserted; and the like In one aspect, target molecules, such as proteins, are purified and directly immobilized on a solid support such as a bead or microtiter plate. This enables the physical separation of bound and unbound phage simply by washing the support. Numerous supports are available for this purpose, including modified affinity resins, glass beads, modified magnetic beads, plastic supports, and the like. Useful supports are those that have low background for nonspecific phage binding and that present the target molecules in a native configuration and at a desirable concentration.

In some embodiments, a nucleic acid-encoded binding compound is an antibody fragment expressed by a phage. In one embodiment, such phage is a filamentous bacteriophage and the antibody fragment is expressed as part of a coat protein. In particular, such phage may be a member of the Ff class of bacteriophages. In a further embodiment, the host of such filamentous bacteriophage is E. coli. In another embodiment, a phagemid-helper phage system is used for displaying antibody fragments. Phagemids may be maintained as plasmids in a host bacteria and phage production induced by further infection with a helper phage. Exemplary phagemids include pComb3 and its related family members, e.g. disclosed in Barbas et al, Proc. Natl. Acad. Sci., 88: 7978-7982 (1991), and pHEN1 and its related family members, e.g. disclosed in Hoogenboom et al, Nucleic Acids Research, 19: 4133-4137 (1991); and U.S. Pat. Nos. 5,969,108; 6,806,079; 7,662,557; and related patents, which are incorporated herein by reference. In a particular embodiment, an antibody fragment is expressed as a fusion protein with phage coat protein g3p.

### Libraries of Nucleic Acid-Encoded Binding Compounds

As mentioned above, a feature of the invention is the use of focused libraries from which reliable values for properties of interest, such as relative affinities, expression levels, stabilities, and the like, can be obtained. In one aspect, this eliminates the need for successive cycles of selection, elution, and amplification, as required in conventional approaches. The size of such focused libraries of candidate binding compounds is influenced by at least two factors: the scale of sequencing required for analyzing selected and non-selected binding compounds, such as, binders and nonbinders, and the difficulty of synthesizing polynucleotides that encode library members. That is, the larger the library of candidate compounds and the higher the degree of confidence desired in the binding statistics of each compound both require that more binders and nonbinders be sequenced. Likewise, a larger library of candidate compounds means a greater number of polynueleotides need to be synthesized.

An experimental quantity that embodies the above trade-off is the coefficient of variation (CV) of the measured value of a property of interest, such as relative affinity of a particular binding compound. In one aspect, library size and sequencing scale are selected so that the values of a property of interest of each binder may be measure with a CV of less than or equal to twenty percent or a CV of less than or equal to ten percent; in another embodiment, such factors are selected so that the values of the property of interest are measured with CVs of less than or equal to five percent; in still other embodiments, such factors are selected so that values arc measured with CVS of less than or equal to two percent. In some embodiments, focused libraries are obtained by varying amino acids in a limited number of locations one or two at a time within a pre-existing binding compound, which may be the same as, or equivalent to, a reference binding compound. Preferably amino acids are varied at different positions one at a time. This is especially useful when an amino acid residue critical for binding is sought to be determined. Thus, for example, members of a library of candidate binding compounds may have nucleotide sequences identical to that encoding the pre-existing binding compound except for a single codon position. At that position, each member will have at least one codon different from that of the pre-existing binding compound. Substituted codons may include synonymous and non-synonymous depending on how a binding compound library is synthesized.

Such libraries may include members having an amino acid deletion at such location and may not necessarily include members with every possible codon at such location. Libraries may contain members corresponding to such substitutions (and deletions) at each of a set of amino acid locations within the pre-existing binding compound. The locations may be contiguous or non-contiguous. In some embodiments, the number of locations, or predetermined sites, where codons are varied are in the range of from 1. to 500; in another aspect, the number of such locations are in the range of from 1 to 250; in other embodiments, the number of such locations are in the range of from 10 to 100; and in still other embodiments, the number of such locations are in the range of from 10 to 250. A pre-existing binding compound may be any pre-existing antibody for which sequence information is available (or cart be obtained). Typically, a pre-existing binding compound is a commercially important binding compound, such as an antibody drug, for which one desires to modify one or more properties, such as solubility, immunogenicity, reduction of cross reactivity, increase in stability, aggregation resistance, or the like, as discussed above. However, libraries for use in the method of the invention may comprise mutants of virtually any protein or peptide capable of participating in a competitive, binding reaction.

In one embodiment, the locations where codons are varied comprise the V_{H} and V_{L} regions of the antibody, including both codons in framework regions and in CDRs; in another embodiment, the locations where codons are varied comprise the CDRs of the heavy and light chains of the antibody, or a subset of such CDRs, such as solely CDR1, solely CDR2, solely CDR3, or pairs thereof. In another embodiment, locations where codons are varied occur solely in framework regions for example, a library of the invention may comprise single codon changes solely from a reference binding compound solely in framework regions of both V_{H} and V_{L} numbering in the range of from 10 to 250. In another embodiment, the locations where codons are varied comprise the CDR3s of the heavy and light chains of the antibody, or a subset of such CDR3s. In another embodiment, the number of locations where codons of V_{H} and V_{L} encoding regions are varied are in the range of from 10 to 250, such that up to 100 locations are in framework regions. In another embodiment, nucleic acid encoded binding compounds are derived from a pre-existing binding compound, such as a pre-existing antibody. Exemplary pre-existing binding compounds include, but arc not limited to, antibody-targeted drugs or antibody-based drugs such as adalimumab (Humira), bevacizumab (Avastin), cetuximab (Erbitux), efalizumab (Raptiva), infliximab (Remicade), panitumumab (Vectubix), ranibuzumab (Lucentis), rituximab (Rituxan), trastuzurnab (Herceptin), and the like.

In seine embodiments, the above codon substitutions are generated by synthesizing coding segments with degenerate codons, inserting one or more “NNN” codons. The coding segments are then ligated into a vector, such as a replicative form of a phage, to form a library. Many different degenerate codons may be used with the present invention, such as the exemplary condons shown in Table I.

TABLE I
Exemplary Degenerate Codons

| Codon* | Description | Stop Codons | Number |
|---|---|---|---|
| NNN | All 20 amino acids | TAA, TAG, TGA | 64 |
| NNK or NNS | All 20 amino acids | TAG | 32 |
| NNC | 15 amino acids | none | 16 |
| NWW | Charged, hydrophobic | TAA | 16 |
| RVK | Charged, hydrophilic | none | 12 |
| DVT | Hydrophilic | none | 9 |
| NVT | Charged, hydrophilic | none | 12 |
| NNT | Mixed | none | 16 |
| VVC | Hydrophilic | none | 9 |
| NTT | Hydrophobic | none | 4 |
| RST | Small side chains | none | 4 |
| TDK | Hydrophobic | TAG | 6 |

*Symbols follow the IUB code: N = G/A/T/C, K = G/T, S = G/C, W = A/T, R = A/G, V = G/A/C, and D = G/A/T.

In some embodiments, the size of binding compound libraries used in the invention varies from about 1000 members to about 1×10⁵ members; in another aspect, the size of libraries used in the invention varies from about 1000 members to about 5×10⁴ members; and in further embodiments, the size of libraries used in the invention varies from about 2000 members to about 2.5×10⁴ members. Thus, nucleic acid libraries encoding such binding compound libraries would have sizes in ranges with upper and lower bounds up to 64 times the numbers recited above.

### Nucleic Acid Sequencing Techniques

As mentioned above, a variety of DNA sequence analyzers are available commercially to determine the nucleotide sequences of binder and non-binders in accordance with the invention. Commercial suppliers include, but are not limited to, 454 Life: Sciences, Helicos, Life Technologies Corp., Illumina, Inc. (which produces sequencing instruments using Solexa-based sequencing techniques), Pacific Biosciences, and the like. Also, DNA sequencing techniques under commercial development may be used for implementing the invention e.g. techniques disclosed in the following references, which are incorporated by reference; Rothberg et al, Nature, 475: 348-352 (2011); Rothberg et al, U.S. patent publication 2009/0026082; Anderson et al, Sensors and Actuators B Chem., 129: 79-86 (2008); Pourmand et al, Proc. Natl. Acad. Sci., 103: 6466-6470 (2006); Rethberg et al, U.S. patent publication 2010/0137143; Mena et al, U.S. patent publication 2009/0029477; and the like. The use of particular types DNA sequence analyzers is a matter of design choice, where a particular analyzer type may have performance characteristics (e.g. long read lengths, high number of reads, short run time, cost, etc.) that are particularly suitable for the experimental circumstances and binding compounds being analyzed. DNA sequence analyzers and their underlying chemistries have been reviewed in the following references, which are incorporated by reference for their guidance in selecting DNA sequence analyzers: Bentley et al, Nature, 456: 53-59 (2008)(describing Solexa-based sequencing); Kircher et al, Bioessays, 32: 524-536 (2010); Shendure et al. Science. 309: 1728-1732 (2005); Margulies et al. Nature, 437: 376-380 (2005); Metzker, Nature Reviews Genetics, 11: 31-46 (2010); Heil et al, Electrophoresis, 29; 4618-4626 (2008); Anderson et al. Genes, 1: 38-69 (2010); Fuller et al, Nature Biotechnology, 27: 1013-1023 (2009); and the like. Generally, nucleic acids of binding compounds are extracted and prepared for sequencing in accordance with instructions of a DNA sequence analyzer's instructions.

### EXAMPLE

### Construction of an Avastin-Based Binding Compound Library and Competition Between Avastin and Library Members for A VEGF Target

An Avastin-based binding compound library was made in a phage display system as described in DuBridge, U.S. patent publications 2012/0077691 and 2012/0258866, which are each incorporated herein by reference for this teaching. Library members were Fab fragments in which a selected set of amino acid locations in the VH chain had been encoded by a single wildcard “NNN” codon, but which were otherwise identical to the corresponding Avastin segment. Concentrations for a competitive binding experiment were determined by initially carrying out, a conventional ELISA in a series of wells of a multi-well plate, wherein each well was coated with an equal amount of the target molecule, or ligand, VEGF (10 ng) followed by to blocking agent (BSA, 3 ug). Competitive binding reactions were carried out in each well (in a row of eight) using the same amount of phage library and different amounts of Avastin. After incubation for 72 hours, the wells were washed, anti-phage antibody/HRP conjugate was added and incubated for 1 hour, after which the wells were washed again and the amount of anti-phage antibody remaining bound in each well was determined using a commercially available enzyme-based signaling system (Ultra-TMB) that produced a colored product having an optical density in the wells proportional to anti-phage antibody concentration. Avastin concentrations for subsequent competitive binding experiments were selected as follows: 1 ug, 300 ng, 100 ng. 30 ng, 10 ng, 3 ng, and 0 ng. In each binding reaction, bound phage were harvested and their heavy chain CDR3 encoding region was amplified and sequenced,

Exemplary results are shown in FIGS. 4A and 4B for substitutions at the F100f amino acid location in the Avastin heavy chain. For each amino acid substitution shown, seven bars represent seven relative frequencies for seven different concentrations of Avastin: from left to right, the bars correspond to the concentrations of 1 ug, 300 ng, 100 ng, 30 ng, 10 ng, 3 ng, and 0 ng.

The methionine mutation (“M” on horizontal axis) looks like a well behaved substitution in this dataset, neutral affinity and competitively inhibited with increasing concentrations of Avastin. Thus, the methionine mutation exhibits monotonically decreasing affinity to the target molecule as the concentration of Avastin is increased, which is illustrated by dashed line (400). In contrast the leucine mutation (“L” on horizontal axis) loses some degree of binding specificity and the tryptophan mutation (“W” on the horizontal axis) becomes much more non-specific. Clearly the methionine variant at this site would be the best choice when designing a biosimilar molecule with equivalent binding specificity. In addition, substitutions that appear to have higher affinity in competition selections like the cysteine and histidine changes (“C” and “H” on the horizontal axis, respectively) seem to be much less well competed by Avastin binding and may have suffered large scale structural changes by its mutation relative to the reference compound.

While the present invention has been described with reference to several particular example embodiments, those skilled in the art will recognize that many changes may be made thereto without departing from the spirit and scope of the present invention. The present invention is applicable to a variety of sensor implementations and other subject matter, in addition to those discussed above.

### Definitions

Unless otherwise specifically defined herein, terms and symbols of nucleic: acid chemistry, biochemistry genetics, and molecular biology used herein follow those of standard treatises and texts in the field, e.g. Komberg and Baker, DNA Replication, Second Edition (W.H. Freeman, N.Y., 1992): Lehninger, Biochemistry, Second Edition (Worth Publishers, New York, 1975); Strachan and Read, Human Molecular Genetics, Second Edition (Wiley-Liss, N.Y., 1999); Abbas et al, Cellular and Molecular Immuology, 6^{th} edition (Saunders, 2007),

“Antibody” or “immunoglobulin” means a protein, either natural or synthetically produced by recombinant or chemical means, that structurally is a member of immunoglobulin superfamily (although it may not be of natural origin) and that is capable of specifically binding to a particular antigen or antigenic determinant, which may be a target molecule as the term is used herein. Antibodies, e.g. IgG antibodies, are usually heterotetrameric glycoproteins of about 150,000 daltons, composed of two identical light (L) chains and two identical heavy (H) chains, as illustrated in FIG. 3. Each light chain is linked to a heavy chain by one covalent disulfide bond, while the number of disulfide linkages varies between the heavy chains of different immunoglobulin isotypes. Each heavy and light chain also has regularly spaced intra-chain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain, as illustrated in FIG. 3. Typically the binding characteristics, e.g. specificity, affinity, and the like, of an antibody, or a binding compound derived from an antibody, are determined by amino acid residues in the V_{H} and V_{L} regions, and especially in the CDR subregions of the V_{H} and V_{L} regions. The constant domains are not involved directly in binding an antibody to an antigen. Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., IgG, IgG₃, IgG₄, IgA1, and IgA₂. “Antibody fragment”, and all grammatical variants thereof, as used herein are defined as a portion of an intact antibody comprising the antigen binding site or variable region of the intact antibody, wherein the portion is free oldie constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodics; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the fight chain variable domain, without an associated heavy chain moiety and (3) single chain polypeptidos containing only one heavy chain variable ion, or a fragment thereof containing the three CDRs of the heavy chain variable region, without an associated light chain moiety; and multispecific or multivalent structures formed from antibody fragments. The term “monoclonal antibody” (mAb) as used herein refers to an antibody obtained from a population of substantially homogeneous antibodies, i.e.,, the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epi topes), each mAb is directed against a single determinant on the antigen.

“Binding compound” means a compound that is capable of specifically binding to a particular target molecule or group of target molecules. Examples of binding compounds include antibodies, receptors, transcription factors, signaling molecules, viral proteins, lectins, nucleic acids, aptamers, and the like, e.g. Sharon and Lis, Lectins, 2^{nd} Edition (Springer, 2006); Klussmann, The Aptamer Handbook: Functional Oligonucleotides and Their Applications (John Wiley & Sons, New York, 2006). In one aspect, binding compounds are proteins, such as antibodies or fragments thereof, receptors, signaling proteins, or the like. Mutants of protein binding compounds, sometimes referred to herein as “binding compound mutants,” “library variants,” or the like, are protein binding compounds that differ from a reference binding compound by one or more amino acid substitutions; in one aspect, each binding compound mutant differs from a reference binding compound by from 1 to 3 amino acid substitutions; and in a further aspect, each binding compound mutant differs from a reference binding compound by one amino acid substitution. As used herein, “antibody-based binding compound” or equivalently “antibody binding compound” means a binding compound derived from an antibody, such as an antibody fragment, including but not limited to, Fab, Fab′, F(ab)₂, and Fv fragments, or recombinant forms thereof. In one aspect, an antibody-based binding compound comprises a scaffold or framework region of an antibody and CDR regions of an antibody. In some embodiments, the binding characteristics of an antibody binding compound (e.g. affinity, specificity, etc.) are determined by such framework and CDR regions and such structures may be expression in various formats, that is, various antibody fragment types and various isotypes.

“Complementary-determining region” or “CDR” means a short sequence (up to 13 to 18 amino acids) in the variable domains of immunoglobulins. The CDRs (six of which are present in IgG molecules) are the most variable part of immunoglobulins and contribute to their diversity by making specific contacts with a specific antigen, allowing immunoglobulins to recognize a vast repertoire of antigens with a high affinity, e.g. Beck et al, Nature Reviews immunology, 10: 345-352 (2010),

“Complex” as used herein means an assemblage or aggregate of molecules in direct or indirect contact with one another. In one aspect, “contact” or more particularly, “direct contact” in reference to a complex of molecules, or in reference to specificity or specific binding, means two or more molecules are close enough so that attractive noncovalent interactions, such as Van der Waal forces, hydrogen bonding, ionic and hydrophobic interactions, and the like, dominate the interaction of the molecules. In such an aspect, a complex of molecules is stable in that under assay conditions, the presence of the complex is thermodynamically favorable. As used herein, “complex” may refer to a stable aggregate of two or more proteins, which is equivalently referred to as a “protein-protein complex.” A complex may also refer to an antibody bound to its corresponding antigen. Complexes of particular interest in the invention are protein-protein complexes and antibody-antigen complexes. As noted above, various types of noncovalent interactions may contribute to antibody binding of antigen, including electrostatic forces, hydrogen bonds, van der Waals forces, and hydrophobic interactions. The relative importance of each of these depends on the structures of the binding site of the individual antibody and of the antigenic determinant. The strength of the binding between a single combining site of an antibody and an epitope of an antigen, which can be determined experimentally by equilibrium dialysis (e.g. Abbas et al (cited above)), is called the affinity of the antibody. The affinity is commonly represented by a dissociation constant (K_{d}), which describes the concentration of antigen that is required to occupy the combining sites of half the antibody molecules present in a solution of antibody. A smaller K_{d} indicates a stronger or higher affinity interaction, because a lower concentration of antigen is needed to occupy the sites. For antibodies specific for natural antigens, the K_{d} usually varies from about 10⁻⁷ M to 10¹¹ M. Serum from an immunized individual will contain a mixture of antibodies with different affinities for the antigen, depending primarily on the amino acid sequences of the CDRs.

“Expression” means the process by which a polypeptide or protein is made using information encoded in a gene or nucleic acid. In one aspect, “expression” means the production of a polypeptide or protein by biological processes that use information encoded in a gene or nucleic acid in accordance with the genetic code. Such biological processes include transcription and translation and are usually carried out in a host organism, or expression host. “Expression system” refers to combinations of expression hosts and expression vectors used with such hosts. A polypeptide or protein produced by an expression system may or may not have a biological function, e.g. binding activity for a target, enzymatic activity, or the like. Expression systems for antibody binding compounds and antibody fragments are well-known in the art, as evidenced by the following references which are incorporated by reference; U.S. Pat. Nos. 7,452,975; 7,892,550; 8,030,023; 6,787,637; 7,329,405; 7,910,104; 7,807,163; 7,947,495; and U.S. patent publication 2010/0322931; and the like.

“Ligand” means a compound that hinds specifically and reversibly to another chemical entity to form a complex. Ligands include, but are not limited to, small organic molecules, peptides, proteins, nucleic acids, and the like. Of particular interest are protein-ligand complexes, which include protein-protein complexes, antibody-antigen complexes, enzyme-substrate complexes, and the like.

“Phage display” is a technique by which variant polypeptides are displayed as fusion proteins to at least a portion of a coat protein on the surface of phage, e.g.. filamentous phage, particles. A utility of phage display lies in the fact that large libraries of randomized protein variants can be rapidly and efficiently selected fur those sequences that bind to a target molecule with high affinity. Display of peptide and protein libraries on phage has been used for screening millions of polypeptides for ones with specific binding properties. Polyvalent phage display methods have been used for displaying small random peptides and small proteins through fusions to either gene III or gene VIII of filamentous phage. Wells and Lowman, Curr, Opin. Struct. Biol., 3:355-362 (1992), and references cited therein. In monovalent phage display, a protein or peptide library is fused to a gene III or a portion thereof, and expressed at low levels in the presence of wild. type gene III protein so that phage particles display one copy or none of the fusion proteins. Avidity effects are reduced relative to polyvalent phage so that selection is on the basis of intrinsic ligand affinity, and phagemid vectors are used, which simplify DNA manipulations. Lowman and Wells, Methods: A companion to Methods in Enzymology, 3:205-0216 (1991).

“Phagemid” means a plasmid vector having a bacterial origin of replication, e.g., ColE.1, and a copy of an intergenic region of a bacteriophage. The phagemid may be used on any known bacteriophage, including filamentous bacteriophage and lambdoid bacteriophage. The plasmid w ill also generally contain a selectable marker for antibiotic resistance. Segments of DNA cloned into these vectors can be propagated as plasmids. When cells harboring these vectors are provided with all genes necessary for the production of phage particles, the mode of replication of the plasmid changes to rolling circle replication to generate copies of one strand of the plasmid DNA and package phage particles. The phagemid may from infectious or non-infectious phage particles. This term includes phagemids, which contain a phage coat protein gene or fragment thereof linked to a heterologous polypeptide gene as a gene fusion such that the heterologous polypeptide is displayed on the surface of the phage particle.

“Phage vector” means a double stranded replicative form of a bacteriophage containing a heterologous gene and which is capable of replication. The phage vector has a phage origin of replication allowing phage replication and phage particle formation. The phage is preferably a filamentous bacteriophage, such as an M13, f1, fd, Pf3 phage or a derivative thereof, or a lambdoid phage, such as lambda, 21, phi80, phi81, 82, 424, 434, etc., or a derivative thereof. The term “phage” my be used in reference to a single stranded form or a double stranded form which from the context will be clear to one of ordinary skill.

“Primer” means an oligonucleotide, either natural or synthetic that is capable, upon forming a duplex with a polynueleotide template, of acting as a point of initiation of nucleic acid synthesis and being extended from its 3′ end along the template so that an extended duplex is formed. Extension of a primer is usually carried out with nucleic acid polymerase, such as a DNA or RNA polymerase. The sequence of nucleotides added in the extension process is determined by the sequence of the template polynueleotide. Usually primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following references that are incorporated by reference: Dieffenbach, editor, PCR Primer: A Laboratory Manual, 2^{nd} Edition (Cold Spring Harbor Press, New York, 2003). “Polypeptide” refers to a class of compounds composed of amino acid residues chemically bonded together by amide linkages with elimination of water between the carboxy group of one amino acid and the amino group of another amino acid. A polypeptide is a polymer of amino acid residues, which may contain a large number of such residues. Peptides are similar to polypeptides, except that, generally, they are comprised of a lesser number of amino acids. Peptides are sometimes referred to as oligopeptides. There is no clear-cut distinction between polypeptides and peptides. For convenience, in this disclosure and claims, the term “polypeptide” will be used to refer generally to peptides and polypeptidcs. The amino acid residues may be natural or synthetic. “Protein” refers to a polypeptide, usually synthesized by a biological cell, folded into a defined three-dimensional structure. Proteins are generally from about 5,000 to about 5,000,000 daltons or more in molecular weight, more usually from about 5,000 to about 1,000,000 molecular weight, and may include posttranslational modifications, such acetylation, acylation, ADP-ribosylation, amidation, disulfide bond formation, farnesylation, demethylation, formation of covalent cross-links, formation of cystine, glycosylation, hydroxylation, iodination, methylation, myristoylation, oxidation, phosphorylation, prenylation, selenoylation, sulfation, and nbiquitination, e.g. Wold, F., Post-translational Protein Modifications: Perspectives and Prospects, pgs. 1-12 in Post-translational Covalent Modification of Proteins, B. C. Johnson, Ed., Academic Press, New York, 1983. Proteins include, by way of illustration and not limitation, cytokines or interteukins, enzymes such as, e.g., kinases, proteases, galactosidases and so forth, protamines, histones, albumins, immunoglobulins, scleroproteins, phosphoproteins, mucoproteins, chromoproteins, lipoproteins, nucleoproteins, glycoproteins, T-cell receptors, proteoglycans, and the like,

“Specific” or “specificity” in reference to the binding of one molecule to another molecule, such as a labeled target sequence for a probe, means the recognition, contact, and formation of a stable complex between the two molecules, together with substantially less recognition, contact, or complex formation of that molecule with other molecules. In one aspect, “specific” in reference to the binding of a first molecule to a second molecule means that to the extent the first molecule recognizes and forms a complex with another molecule in a reaction or sample, it forms the largest number of the complexes with the second molecule. Preferably, this largest number is at least fifty percent. Generally, molecules involved in a specific binding event have areas on their surfaces or in cavities giving rise to specific recognition between the molecules binding to each other. Examples of specific binding include antibody-antigen interactions, enzyme-substrate interactions, formation of duplexes or triplexes among polynucleotides and/or oligonucleotides, receptor-ligand interactions, and the like. As used herein, “contact” in reference to specificity or specific binding means two molecules are close enough that weak noncovalent chemical interactions, such as Van der Waal forces, hydrogen bonding, base-stacking interactions, ionic and hydrophobic interactions, and the like, dominate the interaction of the molecules.

“Wild type” of “reference” or “pre-existing” or “predetermined” in reference to a binding compound are used synonymously to mean a compound which is being analyzed or improved in accordance with the method of the invention. That is, such a compound serves as a starting material from which variant polypeptides are derived, through the introduction of mutations. A “wild type” sequence for a given protein is usually the sequence that is most common in nature, but the term is used more broadly here to include compounds that: have been engineered. Similarly, a “wild type” gene sequence is typically the sequence for that gene which is most commonly found in nature, but the usage here includes genes that may have been engineered from a natural compound, e.g. a gene which has been engineered to consist of bacterial codons even though it encodes a human protein. Mutations may be introduced into a “wild type” gene (and this the protein it encodes) through any available process, e.g. site-specific, mutation, insertion of chemically synthesized segments, or other conventional means. The products of such processes are “variant” or “mutant” forms of the original “wild type” protein or gene. Exemplary reference (or wild type or pre-existing) sequences include antibody-targeted drugs or antibody-based drugs such as adalimumab (Humira), bevacizumab (Avastin), cetuximab (Erbitux), efalizumab (Raptiva) infliximab (Remicade), panitumumab (Vectubix), ranibuzumab (Lucentis), rituximab (Rituxan), trastuzumab (Rerceptin), and the like.

What is claimed is:

1. A method of simultaneously determining competitive binding and target specificity of binding compounds of a library relative to a predetermined reference compound, the method comprising the steps of:
(a) to reacting under binding conditions in a plurality of reactions a predetermined reference compound and a library of binding compounds with a ligand, wherein the predetermined reference compound is present in each reaction at a different concentration and wherein each binding compound is a protein encoded by a linked nucleic acid;
(b) separating in each reaction binding compounds forming complexes with the ligand from binding compounds free of ligand;
(c) sequencing for each reaction nucleic acids of a sample of binding compounds forming complexes with the ligand to determine frequencies of the nucleic acids;
(d) sequencing for each reaction nucleic acids of a sample of expressed binding compounds free of ligand or a sample of expressed binding compounds from the library to determine frequencies of the nucleic acids;
(e) determining affinities of the binding compounds of the samples from each reaction, wherein the affinities are determined by comparing the frequency of nucleotide sequences identified with a binding compound forming complexes with the ligand and the frequency of the same nucleotide sequences identified with the binding compound free of the ligand or with the frequency of the same nucleotide sequences that encode the same binding compound in the library; and
(f) identifying a binding compound as competitively binding with the predetermined reference compound to the ligand whenever the affinities of the binding compound arc a monotonically decreasing function of predetermined reference compound concentration, or identifying a binding compound as non-competitive with the predetermined reference compound in binding to the ligand whenever the affinities of the binding compound are a substantially unchanging function of predetermined reference compound concentration.

2. The method of claim 1 wherein said step of reacting includes reacting said predetermined reference compound and said library with said ligand immobilized an a solid support.

3. The method of claim 2 wherein said step of separating includes removing said binding compounds free of ligand from said binding compounds forming complexes with said ligand.

4. The method of claim 1 wherein each said reactions includes a reaction mixture at is incubated until an equilibrium condition is reached.

5. The method of claim 1 wherein said step of reacting includes reacting said predetermined reference compound and said library with a ligand having a capture moiety.

6. The method of claim 5 wherein said step of separating includes capturing said binding compounds forming complexes by said capture moiety of said ligand.

7. The method of claim 1 wherein said step of reacting take place in a reaction vessel and includes treating the reaction vessel with a blocking agent to reduce nonspecific binding of said binding compounds.

8. The method of claim 1 wherein said samples are selected so that said frequencies are determined with a coefficient of variation of twenty percent or less.

9. The method of claim 8 wherein said library has up to one million distinct members.

10. The method of claim 9 wherein said library has up to one hundred thousand distinct members.

11. The method of claim 1 wherein each of said binding compounds differs from said predetermined reference compound by from 1 to 2 amino acid substitutions selected from 10 to 250 amino acid locations or by 1 amino acid substitution selected from 1 to 500 amino acid locations.

12. The method of claim 1 wherein said ligand is present in each of said reactions at a known concentration.

13. The method of claim 12 wherein said known concentration is the same for each of said reactions.

14. The method of claim 1 wherein said biding compounds are expressed in a phage display system,

15. The method of claim 14 said library of binding compounds in an immunoglobulin format.

16. The method of claim 14 said library of binding compounds m a Fab format.

17. The method of claim 1 wherein said binding compounds are protein display systems.

18. The method of claim 1 wherein said predetermined reference compound is an antibody.
